(19) United States
(12) Patent Application Publication
(10) Pub. No.: US 2020/0011865 A1
(43) Pub. Date: Jan. 9, 2020

(54) METHODS AND KITS FOR DETECTING BASOPHIL ACTIVATION

(71) Applicants: INSERM (Institut National de la Santé et de la Recherche Médicale), Paris (FR); Université Paul Sabatier Toulouse III, Toulouse (FR); Centre National de la Recherche Scientifique, Paris (FR); University of Stanford, Stanford, CA (US)

(72) Inventors: Eric ESPINOSA, Toulouse Cedex 3 (FR); Régis JOULIA, Toulouse Cedex 3 (FR); Salvatore VALITUTTI, Toulouse Cedex 3 (FR); Kaori MUKAI, Stanford, CA (US); Mindy TSAI, Stanford, CA (US); Nicolas GAUDENZIO, Stanford, CA (US); Stephen GALLI, Stanford, CA (US)

(21) Appl. No.: 16/470,004

(22) PCT Filed: Dec. 15, 2017
(86) PCT No.: PCT/EP2017/082964

§ 371 Date: Jun. 14, 2019

(30) Foreign Application Priority Data
Dec. 16, 2016 (EP) 16306704.4

(51) Int. Cl. G01N 33/569; G01N 33/50

(52) U.S. Cl. CPC G01N 33/56972; G01N 33/5047; G01N 2333/70539; G01N 2800/60; G01N 2333/70535; G01N 2800/24; G01N 2800/52; G01N 2333/70596

(57) ABSTRACT

The present invention relates to methods and kits for detecting basophil cells activation. The inventors showed that fluorescent avidin binds to basophil cell surface upon degranulation and that this probe can be used to monitor basophil degranulation More specifically the present invention relates to methods for monitoring of basophil degranulation using avidin-based probes. The method of the invention described here allows to measure direct basophil degranulation following FcεRI crosslinking with allergen. This method provides a direct measurement of degranulation by staining exteriorized granules and unambiguously detects activated basophils degranulated. The extent of the degranulation can be directly deduced from the intensity of fluorescence of fluorochrome-labelled avidin measured on basophils. When applied to allergic patient samples the avidin-based method detected efficiently specific basophil responses.

### FIELD OF THE INVENTION

The present invention relates to methods and kits for detecting basophil activation. More specifically present invention relates to methods and kits for monitoring of basophil degranulation using avidin-based probes.

### BACKGROUND OF THE INVENTION

Basophils are multifunctional effector cells involved in allergic and inflammatory reactions (1-3). These cells are characterized by a cytoplasm filled with granules where are stored several biologically active mediators such as histamine or platelet activating factor. Basophils express the tetrameric high affinity IgE receptor FcεRI on their surface which allows them to bind circulating IgE. Aggregation of the IgE/FcεRI complexes by allergen triggers the degranulation process which is completed when granules are exteriorized and stored mediators released in the medium.

Basophil degranulation is classically monitored by measuring the amount of some granule mediators (such as histamine) released in the cell supernatant. Alternative methods based on flow cytometry were developed more than 30 years ago to assess basophil degranulation at the single cell level (4-8). Derived from those pioneering methods are presently used and commercialized basophil activation tests (BATs). Most of these BATs rely on the exposure of CD63 or on the upregulation of CD203c on the basophil surface upon allergen challenge (9). Yet, these assays do not provide a direct measure of the granule exteriorization process and their measurements are not always correlated with histamine release (4).

Accordingly, there remains an unmet need in the art for specific and more sensitive test for basophils activation, reflecting directly the degranulation process of these cells.

The inventors therefore set up a method that allows us to measure the final step of the degranulation process (granule exteriorization) in individual basophils. This method allows to monitor degranulation by microscopy or flow cytometry at the single-cell level and relies on the basophil granule matrix properties.

In a previous study, they have shown that mast cell degranulation can be monitored by using fluorescent avidin, a probe that selectively binds to heparin, the main granule matrix component (10). Because a substantial part of the exteriorized granules remains on the cell surface following degranulation, avidin-based fluorescent probe binds to the degranulated cells and allows to monitor degranulation by flow cytometry (12). The basophil granule matrix was less investigated than its mast cell counterpart but is known to be composed of highly sulfated or ‘heparin-like’ glycosaminoglycan (12, 13). Moreover, like mast cells, human basophils exteriorize membrane-free granules from multiple openings in their plasma membranes upon stimulation and extruded granules frequently remain adherent to the cell membrane. (14)

Inventors show in this study that fluorescent avidin binds to basophil cell surface upon degranulation and that this probe can be used to monitor basophil degranulation. This new assay is comparable to the CD63 exposure-based BAT but has the advantage to provide a measure that is directly correlated to granule exteriorization.

### SUMMARY OF THE INVENTION

Here inventors show, based on the extensive study of the degranulation process of the basophils, that the fluorescent avidin can efficiently be used to monitor blood basophil degranulation. Inventors show that the method of the invention described here allows to measure direct basophil degranulation following FcεRI crosslinking with allergen. This method provides a direct measurement of degranulation by staining exteriorized granules and unambiguously detects activated basophils degranulated. Additionally, inventors investigate the staining of fluorescent avidin with other white blood cells populations (FcεRI^{low} monocytes and CD203c⁻ CD123⁺ FcεRI⁺ plasmacytoid dendritic cells (pDC)) and 2 other granulocyte populations (neutrophils (FSC^{high} SSC^{high} CD16^{high}) and eosinophils (FSC^{high} SSC^{high} CD16⁺ FcεRI⁺)) and they demonstrate that the method of the invention described here is very specific for basophil activation. Furthermore, the magnitude of the degranulation can be directly deduced from the intensity of fluorescence of fluorochrome-labelled avidin measured on basophils. Finally, inventors show that when applied to allergic patient samples the avidin-based method detected efficiently specific basophil responses. In a second study, inventors report herein that avidin-based fluorescent probe (Av.A488) also can be used to detect activated basophils in whole blood of anonymous blood donors (allergy status unknown) or subjects suffering from allergies (peanuts allergy), and can quantify the extent of such basophil activation. Basophil's markers used in this study are CD123+ and HLA-DR negative markers.

Thus, the present invention relates to a method for detecting/monitoring basophil activation in a fluid sample comprising the steps of i) adding an allergen extract and ii) detecting the cell surface expression of CD123 and FcεRI markers or CD123 and CD203c markers or CD123 and HLA-DR negative markers among the cell population contained in the fluid sample and iii) detecting degranulation of the cell population contained in the fluid sample using avidin-based fluorescent probe iv) concluding that the cells expressing CD123 and FcεRI markers or CD123 and CD203c markers or CD123 and HLA-DR negative markers which are bound by the avidin-based fluorescent probe are activated basophils.

In a final aspect, the invention relates to a kit comprising means for detecting the cell surface expression of CD123 and FcεRI markers or CD123 and CD203c markers or CD123 and HLA-DR negative markers on a cell population and avidin-based fluorescent probe.

### DETAILED DESCRIPTION OF THE INVENTION

### Method for Detecting/Monitoring Basophil Cells Activation

An object of the present invention relates to method for detecting/monitoring basophil activation in a fluid sample comprising the steps of i) adding an allergen extract and ii) detecting the cell surface expression of CD123 and FcεRI markers or CD123 and CD203c markers or CD123 and HLA-DR-negative markers among the cell population contained in the fluid sample and iii) detecting degranulation of the cell population contained in the fluid sample using avidin-based fluorescent probe iv) concluding that the cells expressing CD123 and FcεRI markers or CD123 and CD203c markers or CD123 and HLA-DR-negative markers which are bound by the avidin-based fluorescent probe are activated basophils.

In one embodiment of a method for detecting/monitoring basophil activation, the step ii) of detecting the cell surface expression of markers and step iii) of detecting degranulation of the cell population are inversed.

The term “basophil” (also called “Basophil granulocyte”) has its general meaning in the art and is intended to describe a subpopulation of white blood cells. Basophils are the least common of the granulocytes, representing about 0.5 to 1% of circulating white blood cells. They are involved in inflammatory reactions during immune response, as well as in the formation of acute and chronic allergic diseases, including anaphylaxis, asthma, atopic dermatitis and hay fever. They can infiltrate tissues and produce histamine and platelet activating factor that induce inflammation, and heparin that prevents blood clotting. Basophils arise and mature in bone marrow. When activated, basophils degranulate to release histamine, proteoglycans (e.g. heparin and chondroitin), and proteolytic enzymes (e.g. elastase and lysophospholipase). Basophils express the tetrameric high affinity IgE receptor FcεRI on their surface which allows them to bind circulating IgE. Aggregation of the IgE/FcεRI complexes by allergen triggers the degranulation process which is completed when granules are exteriorized and stored mediators released in the medium.

Basophil degranulation is classically monitored by measuring the amount of some granule mediators (such as histamine) released in the cell supernatant. Alternative methods based on flow cytometry were developed to assess basophil degranulation at the single cell level (4-8). Derived from those pioneering methods are presently used and commercialized basophil activation tests (BATs). Most of these BATs rely on the exposure of CD63 or on the upregulation of CD203c on the basophil surface upon allergen challenge (9).

In some embodiments, basophils according to the present invention are mammalian basophils, most particularly human basophils.

The term “fluid sample” refers to any sample which is susceptible to contain a population of basophil in suspension. Non-limiting examples include biological fluids such as blood (e.g., peripheral blood or umbilical cord blood), urine, lymph, cerebral spinal fluid, or ductal fluid, or such fluids diluted in a physiological solution (e.g., saline, phosphate-buffered saline (PBS), or tissue culture medium), or cells obtained from biological fluids (e.g., by centrifugation) and suspended in a physiological solution. Other examples of a “fluid sample containing cells” include cell suspensions (in physiological solutions) obtained from bone marrow aspirates, needle biopsy aspirates or biopsy specimens from, for example, lymph node or spleen. In some embodiments, the fluid sample is obtained from bone marrow.

In some embodiments, the fluid sample is a blood sample. The term “blood sample” means a whole blood sample obtained from a subject (e.g. an individual for whom it is interesting to determine whether a population of responders basophils can be identified).

In some embodiments, the fluid sample is a WBC sample. The term “WBC” or “White Blood Cells”, as used herein, also refers to leukocytes population, are the cells of the immune system. All white blood cells are produced and derived from multipotent cells in the bone marrow known as hematopoietic stem cells. Leukocytes are found throughout the body, including the blood and lymphatic system. Typically, WBC or some cells among WBC can be extracted from whole blood by using i) immunomagnetic separation procedures, ii) percoll or ficoll density gradient centrifugation, iii) cell sorting using flow cytometer (FACS). Additionally, WBC can be extracted from whole blood using a hypotonic lysis buffer, which will preferentially lyse red blood cells. Such procedures are known to the expert in the art.

In some embodiments, the fluid sample is a sample of purified basophils in suspension. Typically, the sample of basophils is prepared by immunomagnetic separation methods preformed on a WBC sample. For example, basophil cells are isolated by using antibodies for basophil-associated cell surface markers, CD123 and FcεRI or CD123 and CD203c or CD123 and HLA-DR-negative. Commercial kits, e.g. CD123/FcεRI Basophil Isolation Kit II from Miltenyi Biotech (#130-092-662) or (CD123/CD203c or FcεRI) EasySep Human basophil enrichment kit from Stemcell (#19069) are available.

As used herein, the term “CD123” also known as “interleukin-3 receptor” or “alpha-chain of the interleukin-3 receptor” or “IL-3RA” has its general meaning in the art and refers to a cell-surface receptor typically found on the immune cells to transmit the signal of soluble cytokine interleukin-3. This receptor, found on pluripotent progenitor cells, induces tyrosine phosphorylation within the cell and promotes proliferation and differentiation within the hematopoietic cell lines. It is expressed on the cell membrane of basophils (Han X et al Arch Pathol Lab Med. 2008 May; 132(5):813-9) and plasmacytoid dendritic cell (pDCs) as well as some conventional dendritic cell (cDC) among peripheral blood cells. CD123 is expressed across acute myeloid leukemia (AML) subtypes (Munoz L et al Haematologica. 2001 December; 86(12):1261-9.), including leukemic stem cells (Testa U et al Biomark Res. 2014; 2: 4).

As used herein, the term “FcεRI” also known as “high-affinity IgE receptor”, or “Fc epsilon RI”, is the high-affinity receptor for the Fc portion of immunoglobulin E (IgE), an antibody isotype involved in the allergy disorder and immunity to parasites. FcεRI is a tetrameric receptor complex that binds Fc portion of the ε heavy chain of IgE. It consists of one alpha (FcεRIα—antibody binding site), one beta (FcεRIβ—which amplifies the downstream signal), and two gamma chains (FcεRIγ—the site where the downstream signal initiates) connected by two disulfide bridges. It is constitutively expressed on mast cells and basophils (Pawankar R. Curr Opin Allergy Clin Immunol. 2001 1 (1): 3-6) and is inducible in eosinophils.

As used herein, the term “CD203c” also known as “Ectonucleotide pyrophosphatase/phosphodiesterase family member 3”, or “ENPP3” refers to the protein which belongs to a series of ectoenzymes that are involved in hydrolysis of extracellular nucleotides. These ectoenzymes possess ATPase and ATP pyrophosphatase activities and are type II transmembrane proteins. Expression of the human protein has been detected in uterus, basophils, and mast cells (see Bühring H J et al Blood. 2001 May 15; 97(10):3303-5.). This protein has also been used in conjunction with CD63 as a marker for activated basophils in the Basophil Activation Test for IgE mediated allergic reactions (McGowan E C, Saini S. “Current Allergy and Asthma Reports. (2013) 13 (1)).

In one embodiment of the method of the invention basophils markers are CD123 and HLA-DR-negative cells (or HLA-DR⁻) (see Mukai K, et al. J Allergy Clin Immunol. 2016 Jul. 15).

As used herein, the term “HLA-DR” also known as “Human Leukocyte Antigen—antigen D Related” is an MHC class II cell surface receptor encoded by the human leukocyte antigen complex on chromosome 6 region 6p21.31. The complex of HLA-DR and its ligand, a peptide of 9 amino acids in length or longer, constitutes a ligand for the T-cell receptor (TCR). HLA (human leukocyte antigens) were originally defined as cell surface antigens that mediate graft rejection in HLA-mismatched donors. HLA-DR molecules are upregulated in response to Interferon gamma. During an infection HLA-DR molecules (expressed by antigen presenting cells) are loaded with microbial peptides and presented to T cells in order to select and activate specific T cells.

As used herein, the term “HLA-DR negative” or “HLA-DR⁻” cells means that HLADR marker is not expressed at the cell surface of the population of cells analyzed

Standard methods for detecting the expression of a specific surface marker such as CD123 and FcεRI or CD123 and CD203c or CD123 and HLA-DR-negative on the cell surface (e.g. basophils surface) are well known in the art. Typically, the step consisting of detecting the surface expression of a surface marker (e;g. CD123 and FcεRI or CD123 and CD203c or CD123 and HLA-DR-negative) may consist in using at least one differential binding partner directed against the surface marker, wherein said cells are bound by said binding partners to said surface marker.

As used herein, the term “binding partner directed against the surface marker” refers to any molecule (natural or not) that is able to bind the surface marker with high affinity. The binding partners can be antibodies (either polyclonal or monoclonal), preferably monoclonal antibodies. In another embodiment, the binding partners may be a set of aptamers.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention or a fragment thereof can be prepared and isolated using any technique that provides for the production of antibody molecules by continuous cell lines in culture. Techniques for production and isolation include but are not limited to the hybridoma technique originally; the human B-cell hybridoma technique; and the EBV-hybridoma technique.

The binding partners of the invention such as antibodies or aptamers may be labelled with a detectable molecule or substance, such as preferentially a fluorescent molecule, or a radioactive molecule or any others labels known in the art. Labels are known in the art that generally provide (either directly or indirectly) a signal.

As used herein, the term “labelled”, with regard to the antibody or aptamer, is intended to encompass direct labelling of the antibody or aptamer by coupling (i.e., physically linking) a detectable substance, such as a fluorophore [e.g. fluorescein isothiocyanate (FITC) or phycoerythrin (PE) or Indocyanine (Cy5)]) or a radioactive agent to the antibody or aptamer, as well as indirect labelling of the probe or antibody by reactivity with a detectable substance. An antibody or aptamer of the invention may be labelled with a radioactive molecule by any method known in the art. More particularly, the antibodies are already conjugated to a fluorophore (e.g. FITC-conjugated and/or PE-conjugated).

The aforementioned assays may involve the binding of the binding partners (ie. antibodies or aptamers) to a solid support. The solid surface could be a microtitration plate coated with the binding partner for the surface marker. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, Calif.). According to the invention, methods of flow cytometry are preferred methods for detecting the surface expression of the surface markers (i.e. CD123 and FcεRI or CD123 and CD203c or CD123 and HLA-DR-negative). Said methods are well known in the art. In a specific embodiment, fluorescence activated cell sorting (FACS) may be also used.

As used herein, the term “avidin-based fluorescent probe”, is avidin glycoprotein protein coupled with fluorescent dye. Avidin is a highly cationic 66,000-dalton glycoprotein (Livnah O, et al PNAS (1993) 90:5076-5080) with an isoelectric point of about 10.5. The property of avidin is to selectively bind highly negatively charged proteoglycan such as heparin. This property was first used to stain mast cells in fixed tissues (10) and is now also used to stain mast cell granules during the degranulation process (12) In the context of the present invention avidin is labelled with fluorescent dye (i.e. fluorochrome) which are for example, but not limited to fluorescein (FITC), rhodamine, (sulforhodamine), alexa-488, Texas Red and many others. Such fluorescent dyes are known to the expert in the art.

For example commercial kit of avidin-based fluorescent probe e.g; Avidin-Fluorescein (A821), Avidin-Alexa Fluor 488 (A21370), Avidin Texas Red (A820) from ThermoFisher Avidin-FITC and Avidin Sulforhodamine from Sigma and Avidin-Alexa 488 from life technologies are available

As used herein, the term “allergen extract”or “allergen”, means any substance that can cause an allergy (hypersensitivity disorder of the immune system), such as, but is not limited to, bee stings, penicillin, various food allergies, pollens, animal detritus (e.g., house dust mite, cat, dog and cockroach), mold, and fungal allergens.

Exemple of Allergens extracts are but not limited to: bermuda grass (BAG-G2), orchard grass (BAG-G3), perennial rye grass (BAG-G5), timothy grass (BAG-G6), 6-grass mix (BAG-GX1: Orchard grass, perennial rye grass, Timothy grass, meadow fescue, meadow grass, velvet grass), ragweed mix (BAG-WX1: common ragweed, giant ragweed), all from Bühlmann laboratories and 5-grass mix (Alyostal: Phleum pretense, Dactylis glomerata, Anthoxanthum odoratum, Lolium perenne, Poa pratensis, Stallergenes). See also for a list Allergens extracts the website www.allergen.org/, the official site for the systematic allergen nomenclature that is approved by the World Health Organization and International Union of Immunological Societies (WHO/IUIS) Allergen Nomenclature Sub-committee.

In some embodiments, the method of the invention further comprises a step consisting of determining the level of basophils present in the sample.

### Use of Avidin-Based Fluorescent Probe to Detect Basophil Degranulation and Diagnostic Methods

Finally, inventors confirmed that avidin-based fluorescent probe was a relevant marker for basophils in diseases, inventors applied the method of the invention on blood samples from patients with confirmed grass pollen allergy. Patients' WBC were challenged with an array of allergens and analyzed using the three methods (Avidin, CD63 or CD203c staining). The avidin-based method allowed to efficiently detect patients' response to allergens and provided results comparable to those obtained using CD63 and CD203c staining (FIG. 2A-C). Interestingly, when compared to CD203c staining, avidin binding appeared to be a more sensitive method to evaluate the extent of degranulation in response to different allergens (FIG. 2D). The analysis of avidin rMFI upon different allergen challenges (either single allergens or allergen mixtures) allowed us to establish the response pattern of each patient (FIG. 2E). Prick-tests were carried out using the five grasses mixture and correlated with avidin-based assays (FIG. 2E). Finally, the avidin-based method is a suitable alternative to current methods. Its advantage resides in the fact that avidin directly stains cell-bound granules upon degranulation and that the Av.A488 FI provide a measure of the degranulation magnitude.

Accordingly a further object of the invention relates to the use of avidin-based fluorescent probe as to detect for basophil degranulation. Because avidin binds the negatively charged proteoglycans composing granule matrix, avidin-based fluorescent probe can also be used as a quality control marker to assess the degranulation of activated basophil in vitro.

An additional object of the invention relates to an in vitro method for diagnosing an allergic disease to a given allergen in a subject, comprising the steps of determining in a fluid sample obtained from the subject the level of activated basophil by performing the method of claim 7, ii) comparing the level determined in step i) with a reference value and iii) concluding that the subject suffers from an allergic reaction to the tested allergen when the level determined at step i) is higher than the reference value.

As used herein the term “allergic disease” refers to a hypersensitivity disorder of the immune system toward an allergen. An “allergen” comprises any substance that can cause an allergy, such as, but is not limited to, bee stings, penicillin, various food allergies, pollens, animal detritus (e.g., house dust mite, cat, dog and cockroach), mold, and fungal allergens. Example of allergic diseases include but are not limited to allergic rhinitis, allergic conjunctivitis, allergic asthma, atopic eczema, anaphylaxis, insect sting, drug allergies, food allergies, ocular allergic disease or multiple allergies (such as asthma, eczema and allergic rhinitis together). The allergic disease is seasonal or perennial.

The term “diagnosis” means the identification of the condition or the assessment of the severity of the disease.

A “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the activated basophil level (obtained according to the method of the invention) with a defined threshold value. In one embodiment of the present invention, the threshold value is derived from the activated basophil level (or ratio, or score) determined in a blood sample derived from one or more subjects who are responders (to the method according to the invention). In one embodiment of the present invention, the threshold value may also be derived from activated basophil level (or ratio, or score) determined in a blood sample derived from one or more subjects or who are non-responders. Furthermore, retrospective measurement of the activated basophil level (or ratio, or scores) in properly banked historical subject samples may be used in establishing these threshold values. In a preferred embodiment of the present invention, the threshold value may be determined using a blood sample derived from the same subject without stimulation (internal control)

Reference values are easily determinable by the one skilled in the art, by using the same techniques as for determining the level of activated basophil in fluids samples previously collected from the patient under testing.

For instance, as indicated in the Example 1 section, to analyze the relative increase of MFI (Mean Fluorescence Intensity) for the two staining procedures, inventors took advantage of the two non-responder donors (donors #2 and #17) to set the threshold value of these tests to rMFI=0.1.

An additional object of the invention relates to an in vitro method for determining whether is at risk of having allergic diseases in a subject, comprising the steps of detecting a population of activated basophils by performing one of the methods of the invention, wherein the presence of said population indicates that the subject is at risk of having allergic diseases.

An additional object of the invention relates to an in vitro method for monitoring an allergic disease comprising the steps of i) determining the level of activated basophils in a fluid sample obtained from the subject at a first specific time of the disease by performing one of the methods of the invention, ii) determining the level of activated basophils in a sample obtained from the subject at a second specific time of the disease by performing one of the methods of the invention, iii) comparing the level determined at step i) with the level determined at step ii) and iv) concluding that the allergic disease has evolved in worse manner when the level determined at step ii) is higher than the level determined at step i).

The increase can be e.g. at least 5%, or at least 10%, or at least 20%, more preferably at least 30%.

An additional object of the invention relates to an in vitro method for monitoring the treatment of an allergic disease comprising the steps of i) determining the level of activated basophils in a sample obtained from the subject before the treatment by performing the one of the methods of the invention, ii) determining the level of activated basophils in a sample obtained from the subject after the treatment” by performing one of the methods of the invention, iii) comparing the level determined at step i) with the level determined at step ii) and iv) concluding that the treatment is efficient when the level determined at step ii) is lower than the level determined at step i).

The decrease can be e.g. at least 5%, or at least 10%, or at least 20%, more preferably at least 30%.

Typically, in the embodiments as above described, the fluid sample is a blood sample or a WBC sample.

The term “treatment of an allergic disease” as used therein means several medications that may be used to block the action of allergic mediators, or to prevent activation of cells and degranulation processes. These include antihistamines, glucocorticoids, epinephrine (adrenaline), mast cell stabilizers, and antileukotriene agents are common treatments of allergic diseases (Frieri M. “Mast Cell Activation Syndrome”. Clin Rev Allergy Immunol. (2015)). Anti-cholinergics, decongestants, and other compounds thought to impair eosinophil chemotaxis, are also commonly used. Other immunological therapeutic approach of an allergic disease have been tested like blocking IgE with specific antibody (Omalizumab), with inhibitors of masts cells or cytokine based-immunotherapy (see the reviews Holgate, S T. Nature Reviews Immunology Volume: 8 Issue 3 (2008)). These approaches has required the application of biological agents in the form of blocking monoclonal antibodies, fusion proteins

In one embodiment the treatment of an allergic disease is allergen specific immunotherapy

The term “allergen specific immunotherapy” also known as “desensitization” or “hypo-sensitization” as used herein, refers to a medical treatment for some types of allergies. Immunotherapy involves exposing people to larger and larger amounts of allergen in an attempt to change the immune system's response. It is generally safe and effective for allergic rhinitis, allergic conjunctivitis, allergic forms of asthma, and stinging insects. [Rank, M A; et al. Mayo Clinic Proceedings. 82 (9): 1119-23. (September 2007)]

More specifically Allergen-specific immunotherapy (SIT) is an immune-modifying therapy that has been recommended for the treatment of allergic rhinitis, venom hypersensitivity, some drug allergies and mild bronchial asthma. SIT induces immunological tolerance and the induction of blocking IgG4 antibodies through repeated exposure to allergen(s). After experimental or natural exposure to allergens, SIT decreases the recruitment of mast cells, basophils and eosinophils in the skin, nose, eye and bronchial mucosa. SIT produces an increase in the level of allergen-specific IgA and IgG4 antibodies, and a decrease in the level of allergen-specific IgE antibodies. It also induces CD4+CD25+FOXP3+ TReg cells that produce high levels of IL-10 and/or TGFbeta, two cytokines that are known to attenuate allergen-specific TH2 cell responses. IL-10 suppresses mast-cell, eosinophil and T-cell responses (Wu, K., et al. Cell Mol. Immunol. 4, 269-275 (2007)), and the pleiotropic functions of TGFbeta maintain a diverse and self-tolerant T-cell repertoire, including TReg cells (Wan, Y Y. et al. Immunol. Rev. 220, 199-213 (2007)).

### Kits of the Invention

A further object of the invention relates to kit comprising means for detecting the cell surface expression of CD123 and FcεRI markers or CD123 and CD203c markers or CD123 and HLA-DR-negative markers on a cell population and avidin-based fluorescent probe.

In some embodiments, said means are antibodies. In another embodiment, these antibodies are labelled as above described.

Typically, the kits described above will also comprise one or more other containers, containing for example, wash reagents, and/or other reagents capable of quantitatively detecting the presence of bound antibodies. Preferably, the detection reagents include labelled (secondary) antibodies or, where the antibody raised against CD123, FcεRI, CD203c and HLA-DR is itself labelled, the compartments comprise antibody binding reagents capable of reacting with the labelled antibody. A compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the test sample, a container which contains the antibody(s) used in the assay, containers which contain wash reagents (such as phosphate buffered saline, Tris-buffers, and like), and containers which contain the detection reagent.

Typically, a kit of the present invention will also include instructions for using the kit components to conduct the appropriate methods.

The invention will be further illustrated by the following figures and examples. However, these examples and figures should not be interpreted in any way as limiting the scope of the present invention.

### FIGURES

FIG. 1. Fluorescent avidin allows to monitor basophil degranulation. A-C, flow cytometry analysis of basophil stimulated with anti-IgE Abs. Gating strategy and representative FACS profiles (A). Percentages of av.A488+ or CD63+ basophils (B) and gMFI of gated basophils following avidin or CD63 stainings (C). Percentages in the FACS profiles indicate the frequency of gated cells. Each point represents a donor, bars represent median. Two-tailed paired t-test, ns P>0.05, *P<0.05, ***<0.001, ****<0.0001.

FIG. 2 Analysis of basophil degranulation in allergic patients. A-D, 1×106 WBC were stimulated or not with anti-IgE mAb (2.5 μg/mL) or with indicated allergen preparations. Basophils were gated as in FIG. 1, representative FACS profiles (A), Avidin+ basophil frequency (B), CD63+ basophil frequency (C) and CD203c or avidin fluorescence rMFI following stimulation (D). E, avidin rMFI and Prick test results from tested patients.

FIG. 3 Avidin staining is not always correlated with the CD203c upregulation-based assay. 1×106 Human WBC were stimulated or not with anti-IgE mAb (2.5 μg/mL). Basophils were gated as in FIG. 1. A, Representative FACS profiles of donor #7 and #1. B-C, gMFI of Av. A488 (B) and CD203c (C) staining on basophils (pooled data, n=18), arrows indicate discordant results for donors #1 and #6 (yellow circles). D, rMFI of Av. A488+ (red) and CD203c (green) (pooled data, n=18). Threshold (rMFI=0.1, dashed line) was determined using non responder donors (grey circles). Percentages in the FACS profiles indicate the frequency of gated cells. Each point represents a donor, Arrows indicate donor #13. Two-tailed paired t-test, **<0.01 ***<0.001.

FIG. 4 pDC and FcεRI^{low} monocyte are not stained with avidin upon stimulation. 1×10⁶ Human PWBC were stimulated or not with either PMA/Ionomycin or anti-IgE mAb (2.5 μg/mL). (A) Gating strategy used to isolate pDC and FcεRI^{low} monocyte. Doublets were excluded using FSC-A and FSC-H parameters, then low SSC-A and FSC-A cells were gated; FcεRI^{low} monocyte and CD123⁺ cells were selected. To further separate pDC from basophil, pDC were gated as CD203c⁻ cells. Representative FACS profiles of gated CD123+ pDCs (B) or FcεRI^{low} monocytes (D) for Avidin-A488 staining following stimulation with PMA/Ionomycin or anti-IgE. Percentages of Av.488+ pDCs (C) or FcεRI^{low} monocytes (E) from pooled data n=11. Percentages in the FACS profiles indicate the frequency of gated cells. Each point represents a donor, bars represent median. Two-tailed paired t-test, ns P>0.05.

FIG. 5A small fraction of eosinophils or neutrophils stained dimly positive for avidin following stimulation. 1×10⁶ Human PWBC were stimulated or not with either PMA/Ionomycin or anti-IgE mAb (2.5 μg/mL). (A) Gating strategy used to isolate neutrophils and eosinophils. Doublets were excluded using FSC-A and FSC-H parameters, CD16^{high}/FcεRI⁻ neutrophils and CD16⁺/FcεRI⁺ eosinophils were gated from high SSC-A and FSC-A cells. Representative FACS profiles of gated neutrophils (B) or eosinophil (D) for Avidin-A488 staining following stimulation with PMA/Ionomycin or anti-IgE. Percentages of Av.488+ neutrophils (C) or eosinophils (E) from pooled data n=14. Percentages in the FACS profiles indicate the frequency of gated cells. Each point represents a donor, bars represent median. Two-tailed paired t-test, ns P>0.05, ***<0.001.

### EXAMPLE 1: (INSERM)

### Material & Methods

Reagents. Primary antibodies used for immunostaining: anti-FcεRI efluor® 450 (clone AER-37, eBioscience), anti-CD203c BV510 (2.5 μL per test, clone NP4D6, BD Biosciences), anti-CD123 PE-Cy5 (clone 9F5, BD Biosciences), anti-CD16 Alexa 700 (clone 3G8, Beckman Coulter). Reagents used to stimulate peripheral blood cells were as follows: anti-IgE (clone MH25-1, Santa Cruz), PMA, phorbol 12-myristate 13-acetate and ionomycin (Sigma-Aldrich). Allergens extracts: bermuda grass (BAG-G2), orchard grass (BAG-G3), perennial rye grass (BAG-G5), timothy grass (BAG-G6), 6-grass mix (BAG-GX1: Orchard grass, perennial rye grass, Timothy grass, meadow fescue, meadow grass, velvet grass), ragweed mix (BAG-WX1: common ragweed, giant ragweed), all from Bühlmann laboratories and 5-grass mix (Alyostal: Phleum pretense, Dactylis glomerata, Anthoxanthum odoratum, Lolium perenne, Poa pratensis, Stallergenes). Avidin-Alexa 488 (Invitrogen) or anti-CD63 PE (clone H5C6 BD Biosciences) were used to monitor basophils degranulation. Basophils were purified using EasySep™ Human Basophil Enrichment Kit (#19069, Stemcell™) according to the manufacturer's recommendations.

Blood donors. Blood from randomly selected anonymous donors (allergy status unknown) was obtained from buffy coats or fresh heparined blood (Etablissement Français du Sang). Seven patients (3 men, 4 women, at the age of 19 to 57 years, mean age: 36.6 years, median age: 29 years) with a confirmed grass pollen allergy (positive history of rhinoconjunctivitis and positive skin prick test to grass pollen, 5-grass mix, Stallergenes) were included in this study. Venous blood was collected in 10 mL EDTA tubes. The study was approved by the INSERM national ethics committee (#16-280).

White blood cell preparation. Blood was centrifugated, the plasma discarded and red blood cells were lysed using red blood cells lysis buffer (15 mmol/L NH₄Cl, 1 mmol/L KHCO₃, 10 mmol/L EDTA). After centrifugation, white blood cells were washed in PBS and distributed in 96-well V-bottom plate in Tyrode's buffer (1×10⁶ cells in 100 μL). The cells were adapted to 37° C. for 15 minutes before stimulation.

Cell stimulation for confocal analysis. 1×10⁴ purified basophils were sensitized with human IgE for two hours. Then cells were plated on Poly-D-Lysine (Sigma)-coated Lab-Tek™ chambered coverglass (Nunc) in Tyrode's buffer supplemented with 8 μg mL⁻¹ avidin-sulforhodamine 101 (Av.SRho) and warmed at 37° C. for 20 min. Cells were stimulated with 2.5 μg/mL goat anti-human IgE at time=0. Fluorescence was acquired every 2.3 seconds using Zeiss LSM 710 confocal microscope and ZEN software, environmental chamber (37° C. and 5% CO2), 63× Plan-Apochromat objective (1.4 oil). For some experiments, z-stacks image were acquired with an interval of 0.4 μm.

Cell stimulation for FACS analysis. Cells were stimulated with 100 μL of Tyrode's buffer containing either anti-IgE mAb (final concentration 2.5 μg/ml) or a combination of phorbol 12-myristate 13-acetate and ionomycin (final concentration, 50 ng/ml and 1 μg/ml respectively) or allergen extract (recommended concentration or 1/1000 for 5-grass mix) for 20 minutes at 37° C. Cells were washed and stained with a combination of anti-FcεRI efluor® 450, anti-CD203c BV510, anti-CD123 PE-Cy5, anti-CD16 Alexa 700, avidin-Alexa 488 and anti-CD63 PE diluted in 50 μL FACS buffer (PBS plus 0.1% bovine serum albumin) for 30 minutes at 4° C. data were acquired by flow cytometry on a BD LSR-II cytometer and were analyzed by using FlowJo software (Tree Star, Inc, Ashland, Ore.).

Statistical analysis. Relative MFI was calculated as follow: rMFI=(gMFI of stimulated condition−gMFI of unstimulated condition)/gMFI of unstimulated condition. Positive rMFI threshold was determined using non responder donors (Av. A488⁻, no CD203c or CD63 upregulation). Two-tailed paired Student's t-tests were used for comparing two groups (Prism 5, GraphPad Software). P-value range is indicated: ns>0.05 *P<0.05, **P<0.01, ***P<0.001, ****P<0.0001. Shown flow cytometry profiles and microscopy images are representative of repeated experiments.

### Results

IgE-sensitized basophils were stimulated with anti-IgE Abs. Avidin-sulforhodamine (Av.SRho) was added to the incubation medium to monitor degranulation dynamics by time-lapse confocal laser scanning microscopy. Five minutes after stimulation, basophils underwent morphologic changes (e.g. increase of cell perimeter and cell spreading) while exteriorized granules were detected on the cell surface. The Av.SRho fluorescence intensity (FI) of the degranulating basophil augmented progressively to reach a plateau. Taken together, these results show that the addition of Av.SRho to the culture medium allows to monitor basophil degranulation.

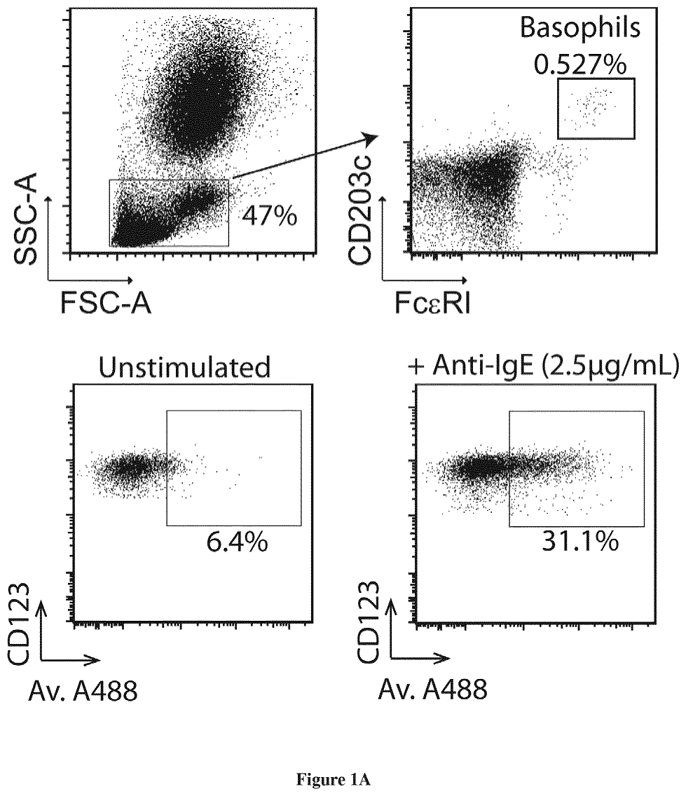

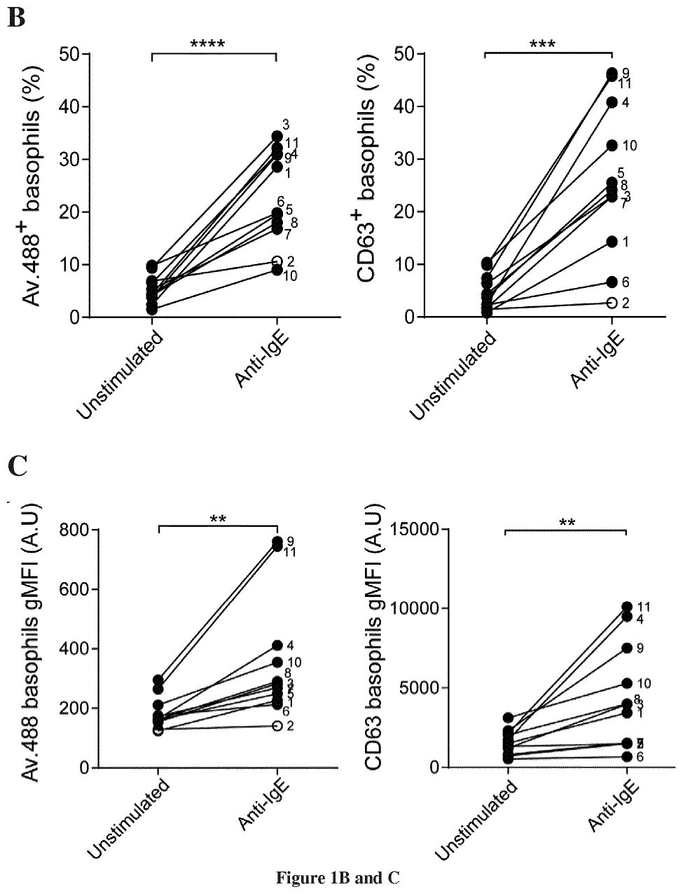

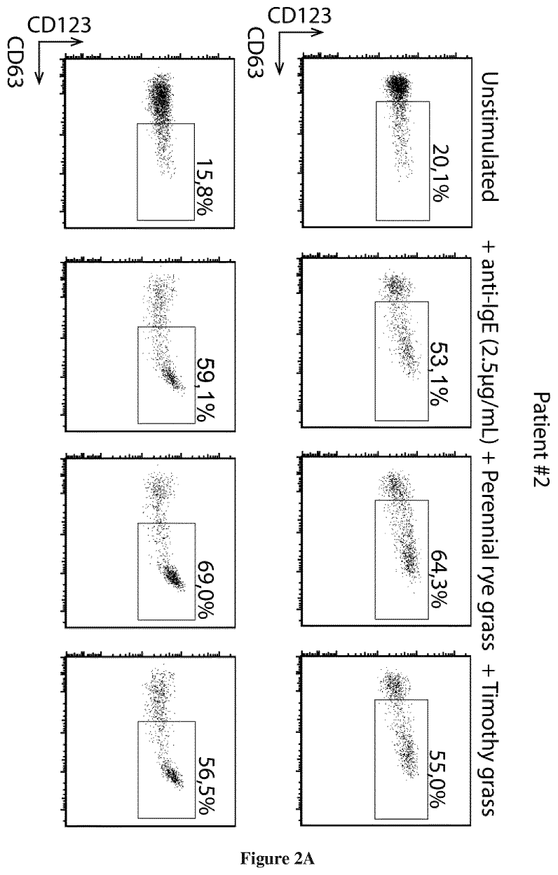

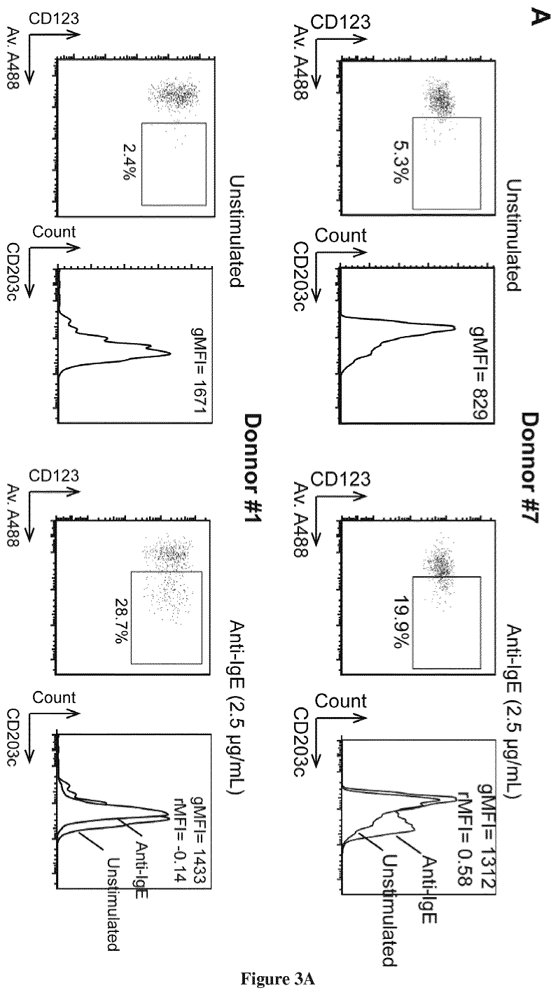

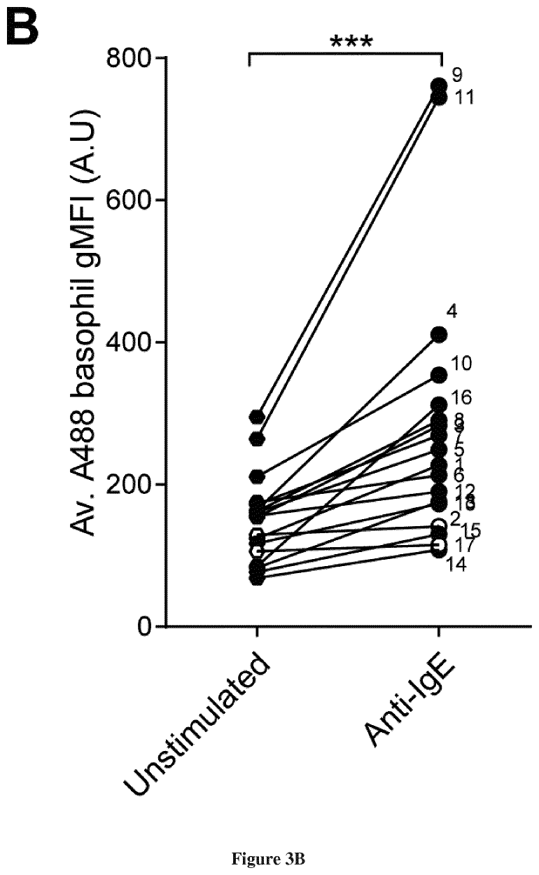

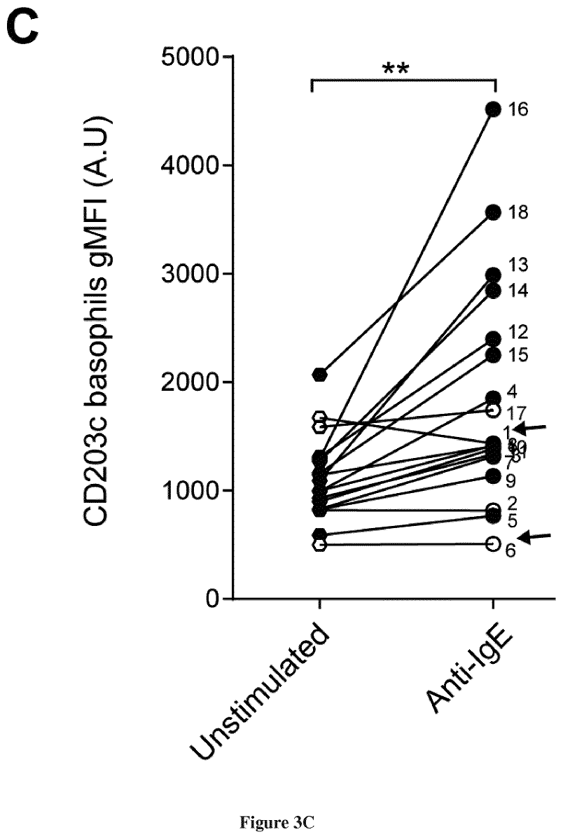

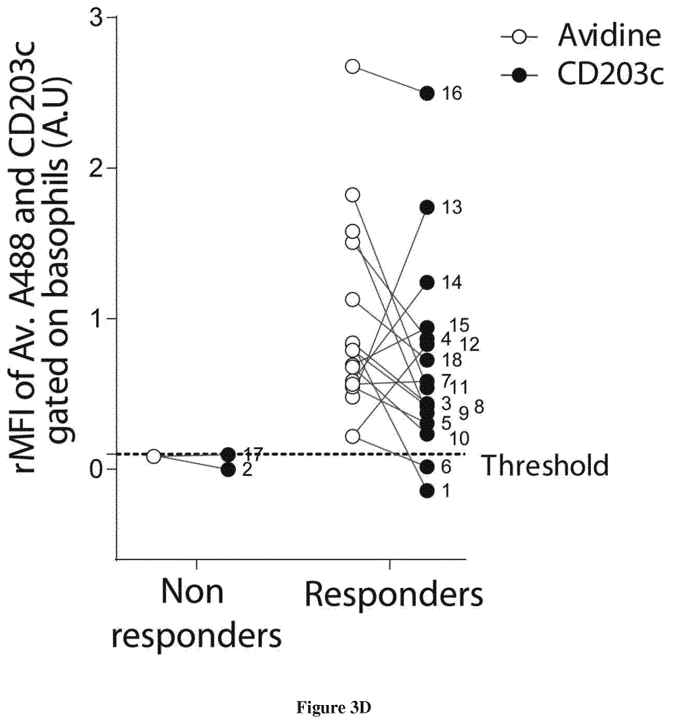

To directly measure basophil specific activation among white blood cells (WBCs), we analyzed fluorescent avidin binding by flow cytometry. Freshly isolated peripheral WBCs from healthy donors were stimulated with anti-IgE Abs. 20 minutes after stimulation, cells were stained with avidin-A488, anti-CD203c, anti-FcεRI, anti-CD123 and anti-CD63 mAbs and analyzed by flow cytometry. Basophils were identified as CD203c⁺ FcεRI⁺ cells. Degranulated basophils stained positive for Av.A488 (FIG. 1A). Analysis of the degranulation as assessed by measurement of CD63 exposure or Av.A488 binding provided similar results, by showing that a substantial fraction of the basophil population degranulated for 10 donors in 11 (FIG. 1B-C). We next compared av.A488 staining to CD203c upregulation on basophil surface following stimulation. Basophils from two donors in 18 did not respond to anti-IgE stimulation as measured by both methods (FIG. 3). Noticeably, we observed a discrepancy among the two methods in two donors (donor #1 and #6) with an increase of avidin staining and no difference in CD203c staining before and after stimulation (FIG. 3). To analyze the relative increase of MFI for the two staining procedures, we took advantage of the two non-responder donors (donors #2 and #17) to set the threshold of these tests to rMFI=0.1. We plotted the relative MFI (rMFI) obtained for all the donors (FIG. 3D). This analysis showed that the rMFI calculated using the avidin-based assay did not correlate with rMFI calculated using the CD203c-based assay. In other words, the two methods do not identify the same donors as high responder donors. The discrepancies observed when comparing CD203c− and avidin-based assays could be explained by the fact that avidin staining is strictly associated to degranulation whereas CD203c is a more generic marker of activation.

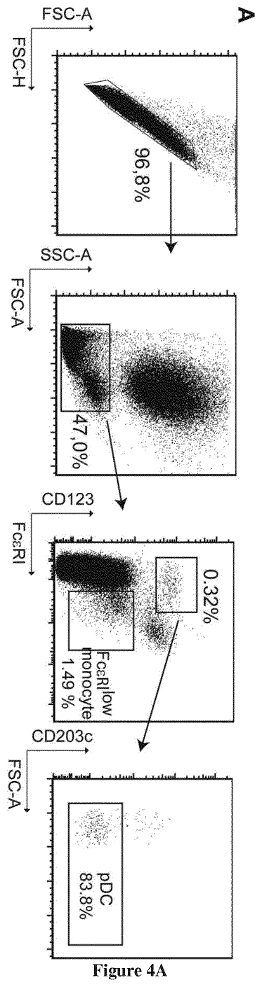

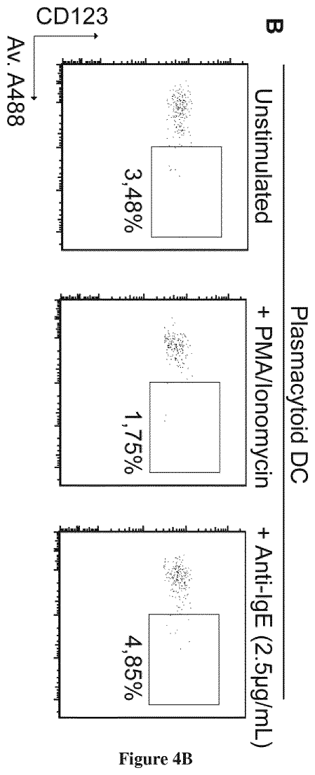

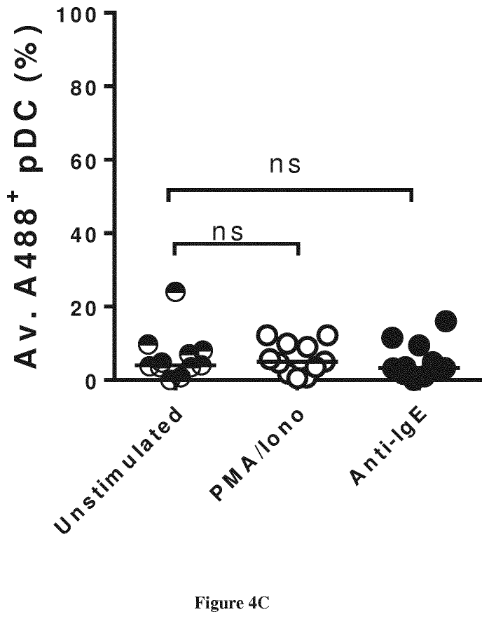

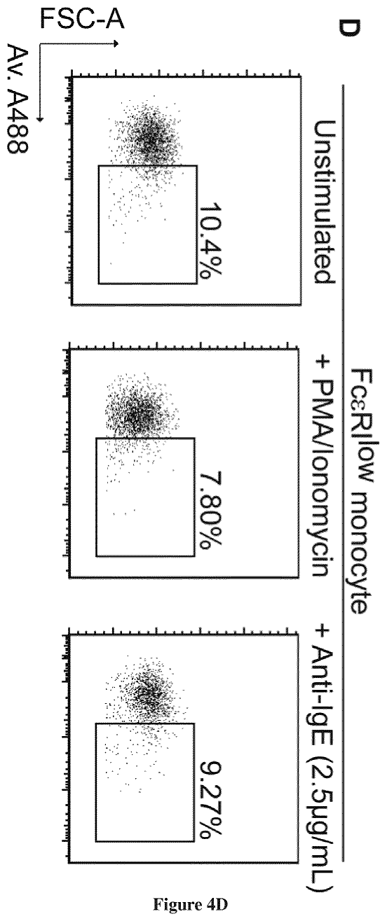

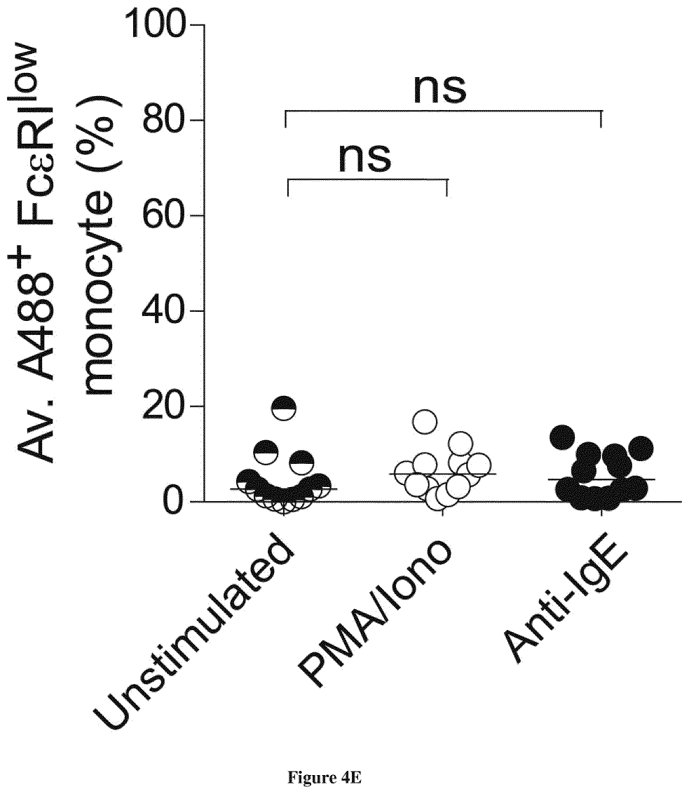

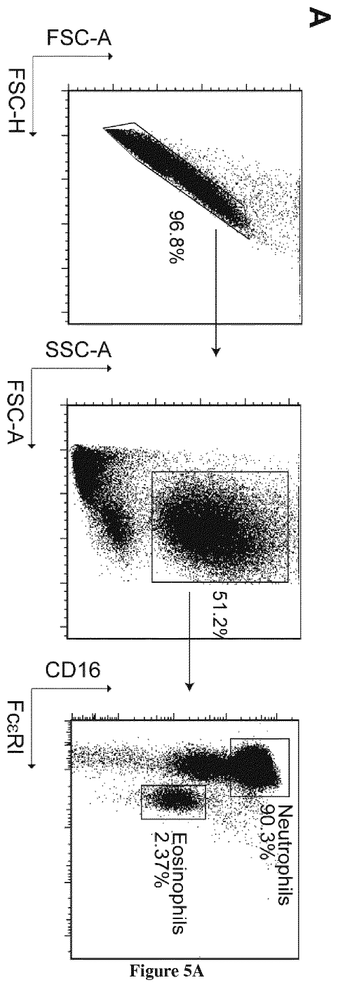

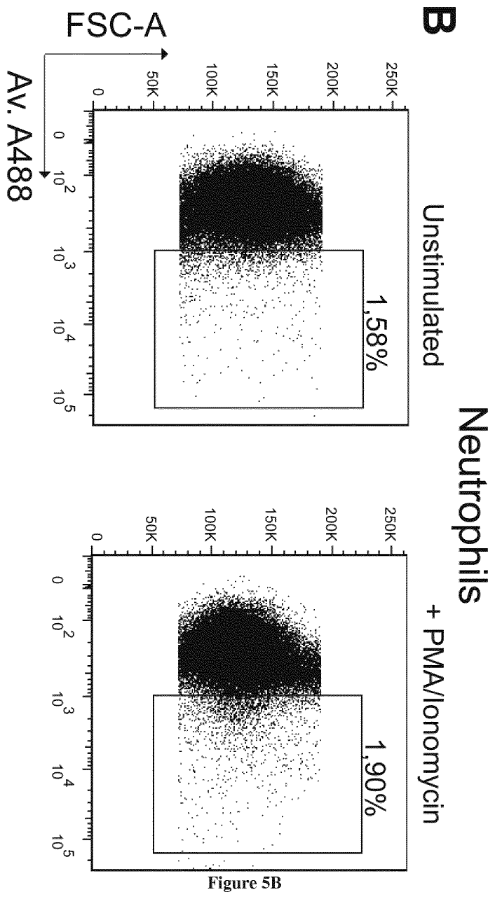

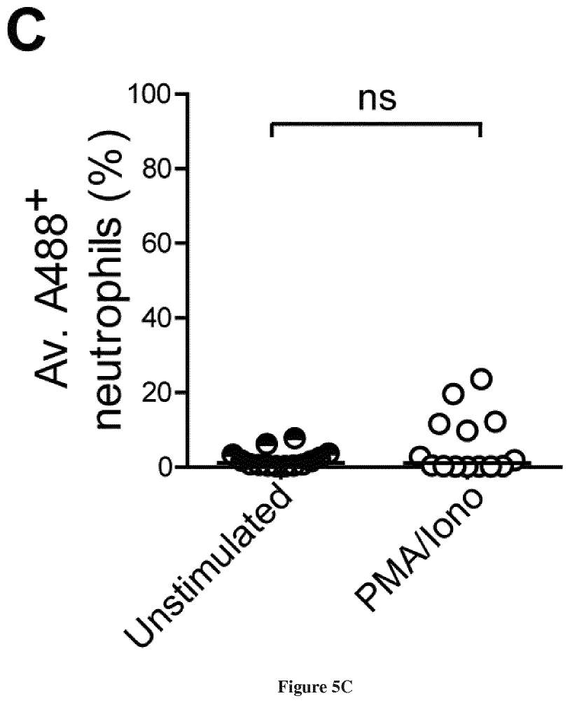

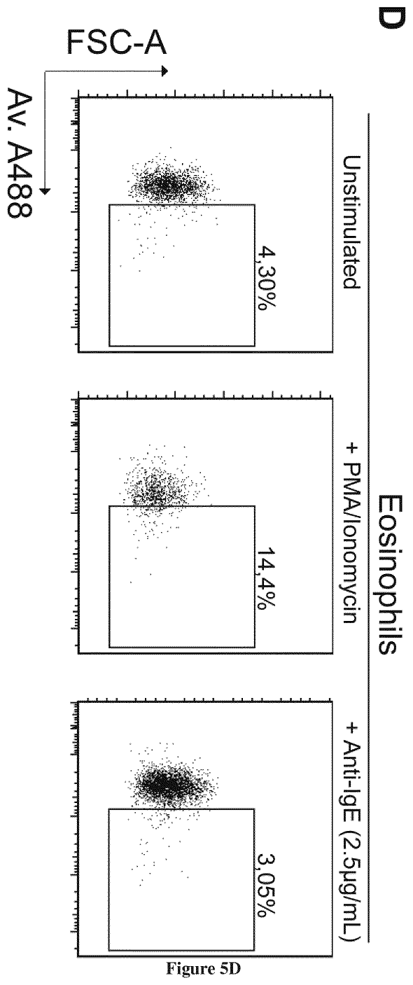

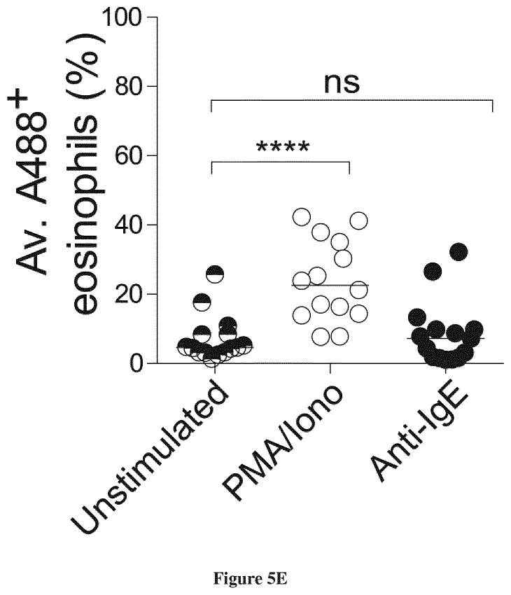

Since other immune cells might be activated following stimulus application to the whole WBC population, we initially investigated whether other cells might bind avidin. In the same set of experiments, we analyzed FcεRI⁺ monocytes and plasmacytoid dendritic cells (pDC) that are present in the low SSC gate. We observed no avidin-A488 staining on FcεRI^{low} monocytes and on CD203c⁻ CD123⁺ FcεRI⁺ pDC (FIG. 4). We in a second approach, we analyzed avidin binding on the two other granulocyte populations, i.e. neutrophils (FSC^{high} SSC^{high} CD16^{high}) and eosinophils (FSC^{high} SSC^{high} CD16⁺ FcεRI⁺) (FIG. 5). We did not detect avidin-A488⁺ neutrophils following stimulation for 9 donors in 14 and a very low percentage of slightly avidin-A488⁺ neutrophils for 5 donors in 14 (FIG. 5B-C). Nevertheless we observed that a small fraction of the eosinophils stained dimly for avidin-A488 for all the donors upon PMA/ionomycin stimulation only (FIG. 5D-E).

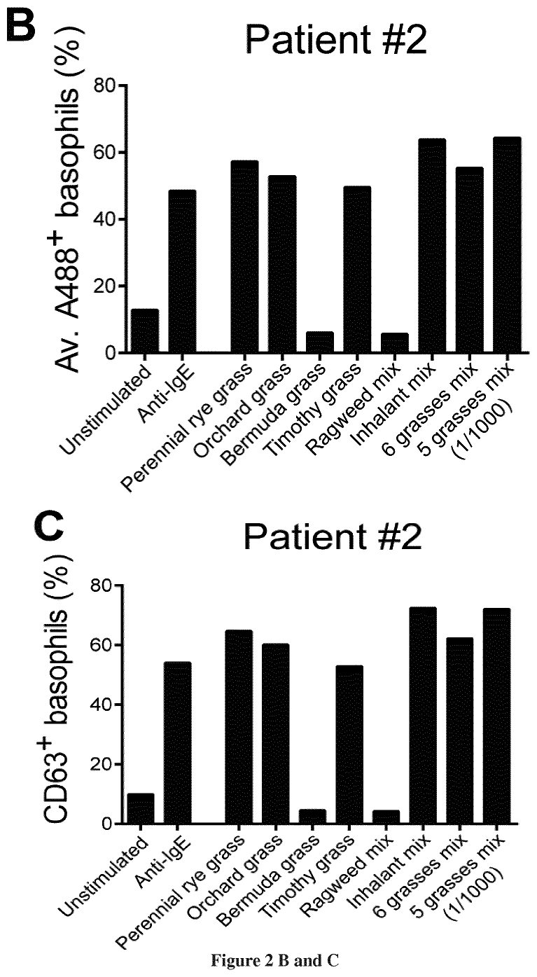

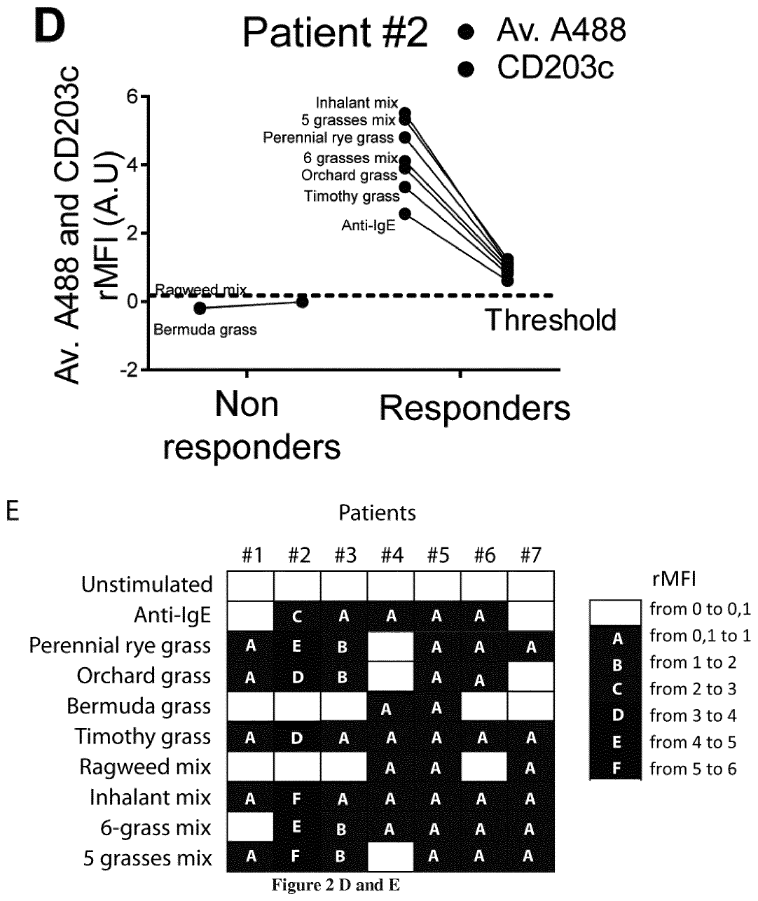

We next tested the avidin-based method on blood samples from patients with confirmed grass pollen allergy. Patients' WBC were challenged with an array of allergens and analyzed using the three methods (Avidin, CD63 or CD203c staining). The avidin-based method allowed to efficiently detect patients' response to allergens and provided results comparable to those obtained using CD63 and CD203c staining (FIG. 2B-D). Interestingly, when compared to CD203c staining, avidin binding appeared to be a more sensitive method to evaluate the extent of degranulation in response to different allergens (FIG. 2D). The analysis of avidin rMFI upon different allergen challenges (either single allergens or allergen mixtures) allowed us to establish the response pattern of each patient (FIG. 2E). Prick-tests were carried out using the five grasses mixture and correlated with avidin-based assays (FIG. 2E).

In conclusion, we described a new test easy to perform that provides similar results when compared to routinely used CD63-based test. It has been reported that CD63 expression does not always correlate with histamine release (5, 13, 14). Accordingly we observed, in mast cells, that degranulation as detected by avidin binding (but not by CD63 staining) correlates with β-hexosaminidase release 12. Additional caveats might apply to CD63-based assays. First, CD63 exposure upon stimulation is not restricted to basophils, underlying the necessity of accurate gating strategies. Second, platelets express CD63 and can bind to basophils upon activation, thus providing a source of potential artifact (4).

Finally, the avidin-based method is a suitable alternative to current methods. Its advantage resides in the fact that avidin directly stains cell-bound granules upon degranulation and that the Av.SRho FI provide a measure of the degranulation magnitude.

### EXAMPLE 2: (STANFORD)

### Methods

### Blood Specimens

Blood from randomly selected anonymous blood donors (allergy status unknown) was obtained from the Stanford Blood Center (Palo Alto, Calif., USA) and blood from 22 peanut allergic patients (Table 1) was obtained as part of their enrollment into two IRB-approved clinical trials (16 from POISED ClinicalTrials.gov Identifier: NCT02103270 and 6 from MAP-X ClinicalTrials.gov Identifier: NCT02643862). All blood samples were collected in heparin tubes and stored at 4° C. for 24 hours with gentle agitation before analysis (9). Peanut allergy was defined as having a clinical reaction to peanut during a double-blind, placebo-controlled food challenge to peanut (up to 500 mg total of peanut protein) and a positive skin prick test to peanut (>= to 5 mm).

TABLE 1
Demographic characteristics of patients with peanut allergy

| | |
|---|---|
| Age (y) | Range 4-26; median = 10, mean = 11 |
| Sex | 82% Male |
| Ethnicity | 95% Not Hispanic/Latino |
| Race* | 68% Caucasian, 27% Asian and 5% multi-racial |

*Subjects self-identified ethnicity and race.

### Reagents

RPMI-1640 medium was purchased from Gibco, Grand Island, N.Y., USA. Polyclonal rabbit anti-human IgE (Bethyl Laboratories, Montgomery, Tex., USA) was used for BATs. The antibody cocktail for surface staining for BATs consisted of FITC-conjugated anti-CD63 mAb (clone: H5C6 from BD Bioscience, San Jose, Calif., USA), PE-conjugated anti-HLA-DR mAb (clone: G46-6 from BD Bioscience), and PerCP-conjugated anti-CD123 mAb (clone: 7G3 from BD Bioscience). Alexa488-conjugated avidin was purchased from ThermoFisher Scientific (Carlsbad, Calif. USA). Calcium/magnesium-free PBS (CMF-PBS) was purchased from Corning Cellgro, Mediatech, Manassas, Va., USA. 0.5 M EDTA was purchased from Invitrogen Life Technologies, Carlsbad, Calif., USA. Bovine serum albumin (BSA) was purchased from Sigma, St. Louis, Mo., USA. Round bottomed tubes (352058) were purchased from BD Falcon, San Jose, Calif., USA. Fixation/Permeabilization Concentrate and Diluent, and Permeabilization Buffer (10×), were purchased from eBioscience, San Diego, Calif., USA. Staining buffer refers to 5% BSA and 2 mM EDTA in CMF-PBS. All reagents were kept sterile at 4° C.

### Basophil Activation Tests

Blood specimens were gently rotated at 4° C. for 24 h after blood collection. Immediately before starting BAT assays, samples were put into a water bath at 37° C. for 30 seconds. 100 μL of whole blood were mixed with 100 μL of RPMI or anti-IgE (final concentration: 1 μg/mL) in 100 μL of RPMI in round-bottomed tubes with loose lids as described in Ref (9). After a 30-min incubation at 37° C. in a 5% CO2 incubator (Panasonic, Osaka, Japan), reactions were stopped by adding 900 μL, of cold 2.5 mM EDTA/CMF-PBS, followed by centrifuging for 5 min at 4° C. (all centrifuge runs were done with these conditions). After removal of supernatants, the antibody cocktail for surface staining (5 μL, of each antibody mentioned above, total 20 μL), or avidin-Alexa488 (final concentration: 1 μg/mL) instead of FITC-labeled CD63 antibody, was added and mixed with the cell pellets, then incubated on ice for 20 min. After incubation, 3 mL of staining buffer was added and the tubes were centrifuged, the supernatant was removed, and 1 mL of Fix/Perm solution was added and mixed, and incubated for 30 min on ice. 2 mL of permeabilization buffer was added after the incubation and the tubes were centrifuged, supernatants were removed, and 150 μL, of staining buffer was added, and then flow cytometry was performed with a FACS Canto II (BD Bioscience). Data were analyzed with FlowJo (TreeStar, Ashland, Ore., USA) by gating basophils as CD123 (IL-3 receptor alpha chain) positive and HLA-DR negative cells, and then measuring expression of and CD63 or avidin positive populations (data not shown).

### Basophil Analysis by Confocal Microscopy

Human basophil enrichment kit (STEMCELL Technologies, Vancouver, Canada) was used for purification of basophils as per manufacturer's instructions. 5×10⁴ Purified basophils were placed into poly-D-Lysine-coated (5 μg/ml in water, #P6407, Sigma Aldrich, USA) Nunc Lab-Tek 1.0 borosilicate cover glass system 8 chambers (#155411, Thermoscientific, USA) in RPMI medium supplemented with 1 μg/ml of Av.A488 in a controlled atmosphere (using a Zeiss stage-top incubation system with objective heater, 37° C. and 5% humidified CO2) (8). Stimuli were added as described above and 30 minutes later single cell images were taken using a Zeiss LSM780 Meta inverted confocal laser-scanning microscope and a 63×/1.40 Oil DIC M27 objective and electronic zoom 3 (dimension x:512 y:512, scaling x=0.264 μm and y=0.264 μm) (8).

### Statistical Analysis

Mann-Whitney U tests were performed (the groups analyzed are described in the figure legends), and the results are reported in figure legends. We considered a P value of less than 0.05 to be statistically significant.

### Result

First, we purified basophils from the whole blood of anonymous blood donors and used single cell confocal imaging to determine whether Av.A488 could stain basophils stimulated by activation with anti-IgE, which results in basophil degranulation. In accord with our previous reports on primary human and mouse MCs, (11, 15) we observed that, compared to RPMI-challenged control basophils, anti-IgE stimulated basophils exhibited enhanced staining with Av.A488, with a pattern of staining consistent with the exteriorization and cell surface association of negatively-charged granule constituents (e.g. chondroitin sulfate- and/or heparan sulfate-containing proteoglycans) (data not shown).

We recently reported a simple protocol which permits BATs to be performed, either by conventional flow cytometry or by Cytometry by Time-of-Flight mass spectrometry (CyTOF), on whole blood stored at 4° C. for up to 24 h before analysis (16). We used those conditions of blood storage to determine whether Av.A488 staining could be used to monitor basophil activation in whole blood from a group of 11 anonymous blood donors. We tested 100 μL of whole blood in which cells were challenged with either 1 μg/mL anti-IgE or RPMI (control), then gated on basophils as CD123-positive and HLA-DR-negative cells (two stable markers that, compared to FcεRI or CD203c, do not vary upon basophil activation, (data not shown), (16) and compared detection of activated basophils by flow cytometry using either FITC-labeled anti-CD63 antibody or Av.A488 (both using the same detection channel; we defined activated basophils as those with a positive fluorescence signal for FITC-labeled anti-CD63 or for Av.488 as compared to the unstained and non-stimulated conditions) (data not shown). We found that Av.A488 readily detected anti-IgE-induced basophil activation in the whole blood of such subjects, whose allergy status is unknown (data not shown). Importantly, no significant increase in Av.A488 staining was observed after anti-IgE challenge of other granulocyte populations, i.e., neutrophils and eosinophils.

We next investigated whether Av.A488 could be used to monitor basophil activation in whole blood from 22 peanut allergic patients, drawn from subjects enrolled in one of two IRB-approved clinical trials (16 from POISED ClinicalTrials.gov Identifier: NCT02103270 and 6 from MAP-X ClinicalTrials.gov Identifier: NCT02643862) whose demographic features are shown in Table 1. Peanut allergy in both trials was defined as having a clinical reaction to a double-blind, placebo-controlled food challenge to peanut (up to 500 mg total of peanut protein) and a positive skin prick test to peanut (>= to 5 mm)·9

[symbol]

Using 100 μL of whole blood per patient, we compared the ability of FITC-labeled anti-CD63 antibody versus Av.A488 to detect activated whole blood basophils by flow cytometry. Compared to CD63 detection, Av.A488 identified a higher percentage of activated basophils in 20 of 22 patients tested, both at baseline (i.e., after RPMI incubation) and after basophil activation by anti-IgE (data not shown). We also compared the detection of Av.A488+ basophils in the whole blood of anonymous blood donors versus subjects suffering from peanut allergies, both at baseline (i.e., after RPMI incubation) and 30 min after anti-IgE treatment. Both groups showed comparable increases in the percentage of detected Av.A488+ basophils after anti-IgE stimulation (data not shown). However, while anonymous blood donors in general harbored a small percentage of Av.A488+ basophils at baseline (global median ˜3.4%), this percentage was significantly increased in the blood of allergic subjects (global median ˜7.1%) (data not shown). Interestingly, in 11 of 22 allergic patients (compared to only 1 of 11 anonymous blood donors), basophils exhibited detectable Av.A488+ structures on their surface without prior in vitro stimulation with anti-IgE. It is therefore tempting to speculate that such Av.A488+ basophils either exhibited low “basal” levels of activation even without antigen stimulation and/or persistent effects of prior, perhaps subclinical, episodes of activation (e.g., related to the oral immunotherapy treatments). Notably, whatever its origin, this “activated status” of blood basophils at baseline in these 11 subjects was not observed by assessment of CD63 (data not shown).

Taken together, these results show that fluorochrome-labelled avidin can detect basophil degranulation and also might represent a more specific and sensitive alternative to CD63 detection to monitor the activation status of blood basophils in the whole blood, either at baseline or after IgE-dependent stimulation ex vivo.

### REFERENCES

Throughout this application, various references describe the state of the art to which this invention pertains. The disclosures of these references are hereby incorporated by reference into the present disclosure.
1. Voehringer D. Basophils in allergic immune responses. Curr Opin Immunol 2011; 23:789-93.
2. Schroeder J T. Basophils: emerging roles in the pathogenesis of allergic disease. Immunol Rev 2011; 242:144-60.
3. Siracusa M C, Kim B S, Spergel J M, Artis D. Basophils and allergic inflammation. J Allergy Clin Immunol 2013; 132:789-801; quiz 788.
4. MacGlashan D W, Jr. Graded changes in the response of individual human basophils to stimulation: distributional behavior of events temporally coincident with degranulation. J Leukoc Biol 1995; 58:177-88.
5. MacGlashan D W, Jr., Bochner B S, Warner J A. Graded changes in the response of individual human basophils to stimulation: distributional behavior of early activation events. J Leukoc Biol 1994; 55:13-23.
6. Nakagawa T, Stadler B M, de Weck A L. Flow-cytometric analysis of human basophil degranulation. I. Quantification of human basophils and their degranulation by flow-cytometry. Allergy 1981; 36:39-47.
7. Pruzansky J J, Zeiss C R, Patterson R. A linear correlation between histamine release and degranulation of human basophils by specific antigen or the ionophore A23187. Immunology 1980; 40:411-6.
8. Knol E F, Mul F P, Jansen H, Calafat J, Roos D. Monitoring human basophil activation via CD63 monoclonal antibody 435. J Allergy Clin Immunol 1991; 88:328-38.
9. MacGlashan D W, Jr. Basophil activation testing. J Allergy Clin Immunol 2013; 132:777-87.
10. Tharp M D, Seelig L L, Jr., Tigelaar R E, Bergstresser P R. Conjugated avidin binds to mast cell granules. J Histochem Cytochem 1985; 33:27-32.
11. Joulia R, Gaudenzio N, Rodrigues M, Lopez J, Blanchard N, Valitutti S, et al. Mast cells form antibody-dependent degranulatory synapse for dedicated secretion and defence. Nat Commun 2015; 6:6174.
12. Joulia R. IL-33 fine-tunes mast cell degranulation and chemokine production at the single cell level. J Allergy Clin Immunol. 2016 Nov. 19. pii: S0091-6749(16)31353-7. doi: 10.1016/j.jaci.2016.09.049. [Epub ahead of print]
13. MacGlashan D, Jr. Expression of CD203c and CD63 in human basophils: relationship to differential regulation of piecemeal and anaphylactic degranulation processes. Clin Exp Allergy 2010; 40:1365-77.
14. Sanz M L, Sanchez G, Gamboa P M, Vila L, Uasuf C, Chazot M, et al. Allergen-induced basophil activation: CD63 cell expression detected by flow cytometry in patients allergic to Dermatophagoides pteronyssinus and Lolium perenne. Clin Exp Allergy 2001; 31:1007-13.
15. Gaudenzio N, Sibilano R, Marichal T, Starkl P, Reber L L, Cenac N, et al. Different activation signals induce distinct mast cell degranulation strategies. J Clin Invest. 2016 Sep. 19. PubMed PMID: 27643442.
16. Mukai K, Gaudenzio N, Gupta S, Vivanco N, Bendall S C, Maecker H T, et al. Assessing basophil activation by using flow cytometry and mass cytometry in blood stored 24 hours before analysis. J Allergy Clin Immunol. 2016 Jul. 15. PubMed PMID: 27527263.

1. A method for detecting/monitoring basophil activation in a fluid sample comprising the steps of i) adding an allergen extract to the fluid sample and ii) detecting the cell surface expression of CD123 and FcεRI markers or CD123 and CD203c markers or CD123 and HLA-DR-negative markers in the cell population contained in the fluid sample iii) detecting degranulation of the cell population contained in the fluid sample using an avidin-based fluorescent probe and iv) concluding that the cells expressing CD123 and FcεRI markers or CD123 and CD203c markers or CD123 and HLA-DR-negative markers which are bound by the avidin-based fluorescent probe are activated basophils.

2. The method according to claim 1 wherein step ii) of detecting the cell surface expression of markers and step iii) of detecting degranulation of the cell population are reversed.

3. The method according to claim 1 wherein the fluid sample is selected from the group consisting of blood samples, bone marrow samples, WBC (white blood cells) samples and samples of basophils in suspension.

4. The method of claim 1 wherein the detection of the cell surface expression of CD123 and FcεRI markers or CD123 and CD203c markers or CD123 and HLA-DR-negative markers on the cell population contained in the fluid sample is performed with a set of antibodies specific for the markers.

5. The method of claim 4 wherein the antibodies specific for the markers are fluorochrome-labelled antibodies.

6. The method of claim 1 wherein the detection of the cell surface expression of CD123 and FcεRI markers or CD123 and CD203c markers or CD123 and HLA-DR-negative markers on the cell population contained in the fluid sample and the detection of the degranulation of the said cell population is performed by flow cytometry.

7. The method of claim 1 which further comprises a step of determining the level of activated basophils present in the sample.

8. (canceled)

9. An in vitro method for diagnosing an allergic disease to a given allergen in a subject, comprising the steps of determining in a fluid sample obtained from the subject the level of activated basophils by performing the method of claim 7, ii) comparing the level determined in step i) with a reference value and iii) concluding that the subject suffers from an allergic reaction to the tested allergen when the level determined at step i) is higher than the reference value.

10. An in vitro method for monitoring an allergic disease comprising the steps of i) determining the level of activated basophils in a sample obtained from the subject at a first specific time of the disease by performing the method of claim 7, ii) determining the level of activated basophil cells in a sample obtained from the subject at a second specific time of the disease by performing the method of claim 7, iii) comparing the level determined at step i) with the level determined at step ii) and iv) concluding that the allergic disease has evolved in a worse manner when the level determined at step ii) is higher than the level determined at step i).

11. An in vitro method for monitoring the treatment of an allergic disease comprising the steps of i) determining the level of activated basophil cells in a sample obtained from the subject before the treatment by performing the method of claim 7, ii) determining the level of activated basophil cells in a sample obtained from the subject after the treatment by performing the method of claim 7, iii) comparing the level determined at step i) with the level determined at step ii) and iv) concluding that the treatment is efficient when the level determined at step ii) is lower than the level determined at step i).

12. The method of claim 11 wherein the treatment is done with an anti-allergic agent or by allergen specific immunotherapy.

13-15. (canceled)
